(19) United States
(12) Patent Application Publication
(10) Pub. No.: US 2026/0207144 A1
(43) Pub. Date: Jul. 23, 2026

(54) PREDICTION OR DETECTION OF MAJOR ADVERSE CARDIAC EVENTS VIA DISRUPTION IN SYMPATHETIC RESPONSE

(71) Applicant: Medtronic Vascular, Inc., Santa Rosa, CA (US)

(72) Inventors: Neil Voskoboynikov, Pembroke Pines, FL (US); Evan J. Stanelle, Shoreview, MN (US)

(21) Appl. No.: 19/134,986

(22) PCT Filed: Nov. 22, 2023
(86) PCT No.: PCT/US2023/080977

§ 371 Date: Jun. 2, 2025

Related U.S. Application Data
(60) Provisional application No. 63/386,581, filed on Dec. 8, 2022.

(51) Int. Cl. A61B 5/00; A61B 5/0205; A61B 5/021; A61B 5/024; A61B 5/026; A61B 5/08; A61B 5/11; A61B 5/145; G16H 40/67; G16H 50/30

(52) U.S. Cl. CPC A61B 5/7275; A61B 5/0205; G16H 40/67; G16H 50/30; A61B 5/021; A61B 5/02405; A61B 5/026; A61B 5/0816; A61B 5/1118; A61B 5/14532; A61B 5/14542

(57) ABSTRACT

This disclosure is directed to systems and techniques for determining a risk that a major adverse cardiac event (MACE) occurs. An example system includes processing circuitry configured to obtain one or more signals indicative of one or more respective physiological parameters and determine values thereof. The processing circuitry is configured to determine a slow-moving average (SMA) and a fast-moving average (FMA) of respective values of the respective one or more respective physiological parameters. The processing circuitry is configured to determine that a respective difference between the respective FMA and the respective SMA meets a respective difference threshold. The processing circuitry is configured to, based on the respective difference meeting the respective difference threshold, at least one of: a) set a flag indicative a risk that a MACE occurs; or b) generate for output an indication of a risk that a MACE occurs.

This application claims the benefit of U.S. Provisional Patent Application 63/386,581, filed Dec. 8, 2022, and entitled “PREDICTION OR DETECTION OF MAJOR ADVERSE CARDIAC EVENTS VIA DISRUPTION IN SYMPATHETIC RESPONSE.”

### TECHNICAL FIELD

The disclosure relates generally to systems and, more particularly, to systems configured to monitor physiological parameters.

### BACKGROUND

Some types of systems may be used to monitor one or more physiological parameters of a patient. These systems may include implantable medical devices (IMDs), wearable devices, or other external devices. Systems may include sensors that sense signals associated with such physiological parameters. A system may utilize sensed physiological parameters to monitor health of a patient.

### SUMMARY

Major adverse cardiac events (MACEs) are usually discovered after a person experiences symptoms and seeks medical help. Sometimes, a MACE is discovered incidentally while person is being imaged or evaluated for other conditions. For example, with onset of myocardial infarction (MI) patients have reported symptoms days or even weeks before being treated for the event. Because it has been shown that “door-to-balloon” time is critical in percutaneous coronary intervention (PCI) related to patient outcomes, early intervention is desirable to reduce the impact of infarction both in the short term and the long term. Therefore, it may be desirable to determine a risk that a person may experience a MACE within a timeframe, which may lead to earlier medical diagnosis and/or treatment and better patient outcomes. Some treatments that may be administered in response to determining a risk that a person may experience a MACE include a PCI, an aortic value or mitral valve replacement or repair, or the like.

A system may be configured to monitor one or more physiological parameters of a person to determine a risk that the person may experience a major adverse cardiac event (MACE) within a timeframe. For example, for a given physiological parameter, the system may monitor the physiological parameter of the person and determine a person-specific homeostasis of the physiological parameter by determining a slow-moving average (SMA) of the physiological parameter. The system may also determine a fast-moving average (FMA) of the physiological parameter. The FMA may be an average, e.g., of samples of the physiological parameter, over a shorter period of time than the SMA. As such, the FMA may be determined based on fewer samples of the physiological parameter than the SMA. For example, the FMA may be based on a subset of samples of the physiological parameters upon which the SMA is based. If the FMA differs by more than (or differs by the same as or more than) a difference threshold from the SMA, the system may generate a flag or an indication for output that is indicative of a risk of a MACE occurring to the patient within the timeframe. In some examples, if the system determines a specific combination of differences between associated FMAs and SMAs of two or more physiological parameters, and differ more than (or are equal to or differ more than) their respective difference thresholds, the system may generate a flag or an indication for output that is indicative of a risk of a MACE occurring to the patient within the timeframe. As used herein, a physiological parameter is a parameter of a biological nature, as opposed to (e.g., not) a parameter of a mechanical nature of a device (e.g., blood flow through a mechanical blood pump) or a parameter based on a parameter of a mechanical nature of a device.

In some examples, a system includes: memory configured to store physiological parameters of a patient; and processing circuitry communicatively coupled to the memory, the processing circuitry being configured to: obtain one or more signals indicative of one or more respective physiological parameters; determine respective values of the one or more respective physiological parameters based on the one or more signals; determine a respective slow-moving average (SMA) of a first subset of the respective values of the one or more respective physiological parameters; determine a respective fast-moving average (FMA) of a second subset of the respective values of the one or more respective physiological parameters; determine that a respective difference between the respective FMA and the respective SMA meets a respective difference threshold; and based on the respective difference meeting the respective difference threshold, at least one of: a) set a flag indicative a risk that a major adverse cardiac event (MACE) occurs; or b) generate for output an indication of a risk that a MACE occurs.

In some examples, a method includes: obtaining, by processing circuitry, one or more signals indicative of one or more respective physiological parameters; determining, by the processing circuitry, respective values of the one or more respective physiological parameters based on the one or more signals; determining, by the processing circuitry, a respective slow-moving average (SMA) of a first subset of the respective values of the one or more respective physiological parameters; determining, by the processing circuitry, a respective fast-moving average (FMA) of a second subset of the respective values of the one or more respective physiological parameters; determining, by the processing circuitry, that a respective difference between the respective FMA and the respective SMA meets a respective difference threshold; and based on the respective difference meeting the respective difference threshold, at least one of: a) setting by the processing circuitry, a flag indicative a risk that a major adverse cardiac event (MACE) occurs; or b) generating by the processing circuitry, for output an indication of a risk that a MACE occurs.

In some examples, a non-transitory computer-readable medium includes instructions, which when executed, cause processing circuitry to: obtain one or more signals indicative of one or more respective physiological parameters; determine respective values of the one or more respective physiological parameters based on the one or more signals; determine a respective slow-moving average (SMA) of a first subset of the respective values of the one or more respective physiological parameters; determine a respective fast-moving average (FMA) of a second subset of the respective values of the one or more respective physiological parameters; determine that a respective difference between the respective FMA and the respective SMA meets a respective difference threshold; and based on the respective difference meeting the respective difference threshold, at least one of: a) set a flag indicative a risk that a major adverse cardiac event (MACE) occurs; or b) generate for output an indication of a risk that a MACE occurs.

This summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 illustrates the environment of an example medical device system in conjunction with a patient, in accordance with one or more techniques of this disclosure.

FIG. 2 is a conceptual drawing illustrating an example configuration of the IMD of the medical device system of FIG. 1, in accordance with one or more techniques described herein.

FIG. 3 is a functional block diagram illustrating an example configuration of the IMD of FIGS. 1 and 2, in accordance with one or more techniques described herein.

FIGS. 4A and 4B are block diagrams illustrating two additional example IMDs that may be substantially similar to the IMD of FIGS. 1-3, but which may include one or more additional features, in accordance with one or more techniques described herein.

FIG. 5 is a block diagram illustrating an example configuration of components of the external device of FIG. 1, in accordance with one or more techniques of this disclosure.

FIG. 6 is a block diagram illustrating an example system that includes an access point, a network, external computing devices, such as a server, and one or more other computing devices, which may be coupled to the IMD of FIGS. 1-4, an external device, and processing circuitry via a network, in accordance with one or more techniques described herein.

FIG. 7 is a flow diagram illustrating example MACE prediction techniques according to one or more aspects of this disclosure.

Like reference characters denote like elements throughout the description and figures.

### DETAILED DESCRIPTION

Certain devices, such as implantable medical devices (IMDs), wearable devices, or other devices, may sense and/or monitor physiological parameters of a person, such as a patient. Such physiological parameters may be indicative of a state-of-health of the person.

As discussed above, MACEs are usually discovered after a person experiences symptoms and seeks medical help. However, depending on the nature of the MACE, the event may not manifest symptoms or may be transient and not confirmed while in the clinic/hospital setting at a given time even after symptoms have presented. Therefore, a system that is configured to sense, identify, and/or log cardiac events while the person is ambulatory may assist clinicians with direction of proper care.

MACEs may be caused by coronary issues, structural issues, and/or conductive issues. Discriminating between the potential causes of a MACE may be time consuming and expensive as each potential type of cause may require different types of testing. For example, detection of structural heart issues may entail costly or invasive echocardiograms. Detection of coronary issues may entail invasive angiograms. Detection of a conductive issue may entail the use of a 12-lead electrocardiogram. Each of these techniques may require capital equipment and/or instrumentation in a hospital or clinical setting.

As such, a system that is configured to determine whether a predicted MACE is more likely to be due to coronary, structural, or conductive issue(s) may be desirable. A system that may do so while a patient is ambulatory may also be desirable. Such a system may be used to avoid unnecessary clinical diagnostic testing or to test a most likely cause first, which may lead to faster diagnosis and/or treatment and reduce the unnecessary use of clinical resources.

This disclosure describes techniques for determining a risk of a MACE occurring to a person. In some examples, the techniques may determine a risk of a MACE within a specified timeframe and/or caused by a specific type of issue, e.g., a structural, coronary, and/or conductive issue. In some examples, a system may generate a flag, and/or generate an indication for output which may include an indication of the risk that the person may experience a MACE, for example, within a specified timeframe. Such a flag or an indication may provide an opportunity for earlier diagnosis and/or treatment of a medical issue which may improve patient outcomes. In some examples, the flag or indication may be indicative of a risk of a MACE within a timeframe such as within x amount of days, y amount of hours, or between a and b amount of days or c and d amount of hours. In some examples, the system may output (e.g., via communication circuitry and/or a user interface) the flag or the indication. In this manner, a patient and/or clinician may become aware that there is a risk of a MACE and may proactively seek or administer testing and/or treatment, thereby improving patient outcomes.

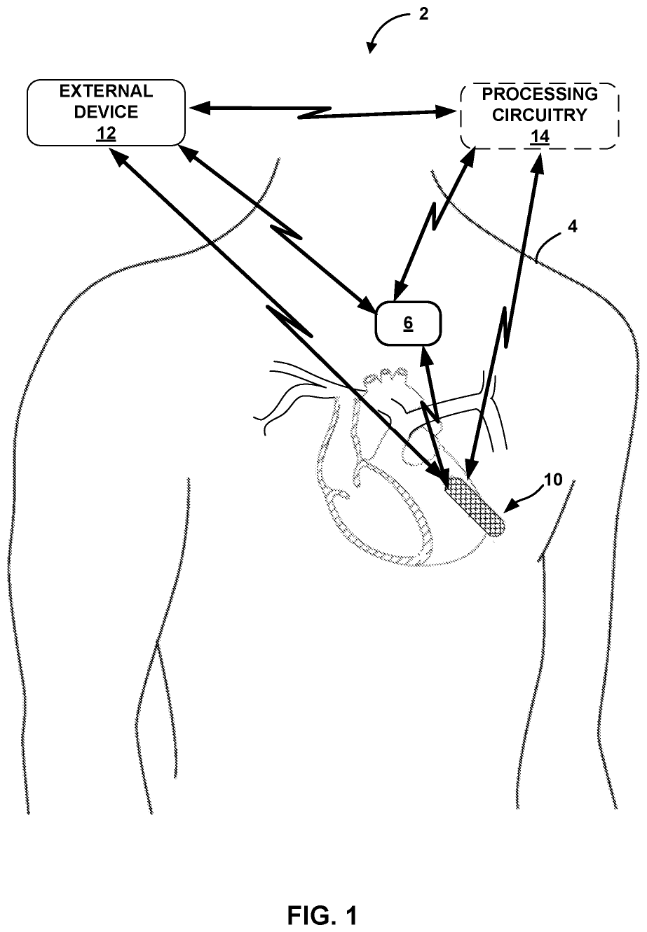

FIG. 1 illustrates the environment of an example medical device system 2 in conjunction with a patient 4, in accordance with one or more techniques of this disclosure. While the techniques described herein are generally described in the context of an ICM, a wearable device, and/or an external device, the techniques of this disclosure may be implemented in any IMD, wearable device, or external device, or combination thereof, capable of sensing and/or processing one or more physiological parameters of patient 4. The example techniques may be used with an IMD 10 and/or wearable device 6 (e.g., a wearable patch), which may be in wireless communication with each other, and/or at least one of external device 12 and/or other devices not pictured in FIG. 1. Processing circuitry 14 is conceptually illustrated in FIG. 1 as separate from IMD 10, wearable device 6, and external device 12, but may be processing circuitry of IMD 10, wearable device 6, and/or processing circuitry of external device 12. In general, the techniques of this disclosure may be performed by processing circuitry 14 of one or more devices of a system, such as one or more devices that include sensors that provide signals, or processing circuitry of one or more devices that do not include sensors, but nevertheless process signals using the techniques described herein. For example, another external device (not pictured in FIG. 1) may include at least a portion of processing circuitry 14, the other external device configured for remote communication with IMD 10, wearable device 6, and/or external device 12 via a network.

In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. For other medical conditions, IMD 10 may be implanted in other appropriate locations, such as the interstitial space, abdomen, back of arm, wrist, etc. In some examples, IMD 10 takes the form of a LINQ™ Insertable Cardiac Monitor (ICM), available from Medtronic plc, of Dublin, Ireland.

In some examples, wearable device 6 may be removably attached to patient 4, such as skin of patient 4, through adhesive, a strap, or other attachment mechanism. For example, wearable device 6 may take the form of a patch, a watch, a wristband, a headband, a chest strap, a mask, a finger clip, a ring, or the like. In some examples, wearable device 6 may be a smart device. In some examples, wearable device 6 may be positioned near (e.g., over) the heart of patient 4.

Clinicians sometimes diagnose patients with medical conditions based on one or more observed physiological signals collected by physiological sensors, such as electrodes, optical sensors, chemical sensors, temperature sensors, acoustic sensors, motion sensors, or the like. In some cases, clinicians apply non-invasive sensors (e.g., wearable sensors) to patients to sense one or more physiological signals while a patient is in a clinic for a medical appointment. However, in some examples, physiological markers, such as those indicative of a risk of patient 4 having a MACE are rare or are difficult to observe over a relatively short period of time. As such, in these examples, a clinician may be unable to observe the physiological markers needed to diagnose a patient with a medical condition or effectively treat the patient while monitoring one or more physiological signals of the patient during the course of a medical appointment.

In the example illustrated in FIG. 1, IMD 10 is implanted within patient 4 to continuously record one or more physiological signals which may be indicative of physiological parameters of patient 4. Such physiological parameters may include, but are not limited to, 1) glucose levels and derivatives, such as time in range; 2) respiration rate; 3) pulse oximeter parameters, such as SpO2 blood oxygen saturation and/or perfusion index; 4) heart rate and derivatives such as heart rate variability, and/or night heart rate; 5) blood pressure and derivatives, such as pulse pressure, mean and/or radial arterial pressure, and/or central venous pressure; 6) physiological parameters discernable from acoustic signals, such as physiological parameters related to rate of blood flow and/or a pulse wave velocity; and/or 7) activity level. Changes in such physiological parameters may be indicative of a risk of a MACE occurring within a timeframe.

In some examples, IMD 10 and/or wearable device 6 includes one or more sensors configured to sense signal(s) indicative of such physiological parameters. For example, such sensors may be configured to detect signals that enable processing circuitry 14, e.g., of IMD 10 and/or wearable device 6, to monitor and/or record physiological parameters of patient 4. For example, IMD 10 and/or wearable device 6 may include a plurality of electrodes, one or more optical sensors, accelerometers, temperature sensors, chemical sensors, light sensors, pressure sensors, audio sensors, and/or respiratory sensors, in some examples. Such sensors may sense one or more physiological parameters indicative of a patient state-of-health. In some examples, additional sensors may be located on other devices (not shown in FIG. 1) which may also sense physiological parameters of patient 4.

Sensor data may be collected by various devices such as implantable therapy devices, implantable monitoring devices, wearable devices, point of care devices, and noncontact sensors in the home or vehicle or other area frequented by the patient or a combination of such sensor platforms. The sensor data collected may be associated with physiological parameters and be relevant to a disease state of patient 4 (e.g., heart failure), comorbidities (e.g., chronic obstructive pulmonary disease (COPD), kidney disease, etc.), or potential issues which may lead to a MACE (e.g., structural, coronary, or conductive).

Processing circuitry 14 may be configured to receive sensed signal(s) indicative of physiological parameter(s) of patient 4, for example, from sensing circuitry of IMD 10 and/or wearable device 6. In some examples, processing circuitry 14 may process one or more of the sensed signals and determine a person-specific (e.g., specific for patient 4) homeostasis of one or more biological parameters of the person. For example, processing circuitry 14 may determine values associated with each of the one or more physiological parameters of patient 4 over time. Processing circuitry 14 may determine a SMA of the values of a given physiological parameter. This SMA may be a mean, median, or mode of the values of the biological parameter(s) calculated over a period of time (an SMA period). In some examples, this SMA period may be a range measured in seconds, hours, days, months, or years. In some examples, when determining an SMA for more than one physiological parameter, the SMA period for each of the physiological parameters may be the same. In other examples, when determining an SMA for more than one physiological parameter, the SMA period for at least one of the physiological parameters may be different than the SMA period for at least one of the others.

Processing circuitry 14 may also determine an FMA of the values of a given physiological parameter. In some examples, the FMA may be a mean, median, or mode of the values of the biological parameter(s) calculated over a period of time (an FMA period). This FMA period may be shorter than the SMA period of the same physiological parameter. In some examples, this FMA period may be a range measured in seconds, hours, days, months, or years. In some examples, when determining an FMA for more than one physiological parameter, the FMA period for each of the physiological parameters may be the same. In other examples, when determining an FMA for more than one physiological parameter, the FMA period for at least one of the physiological parameters may be different than the FMA period for at least one of the others.

For each physiological parameter for which processing circuitry 14 determines a difference between an SMA and a corresponding FMA, a difference threshold may be physiological parameter specific. In other words, a difference threshold may be different for one physiological parameter than for another. In some examples, any of the difference thresholds may be the same as, or different from, any other difference threshold. In some examples, the difference threshold(s) may be programmable. For example, a clinician may program a given difference threshold. For example, if a clinician was more concerned about an acute condition, the clinician may set a relatively higher difference threshold than if the clinician was concerned about a chronic condition, for which the clinician may set a relatively lower difference threshold.

In some examples, the programmability of the difference threshold(s) may be restricted to be of fixed values (e.g., not a value that changes based on the SMA or the FMA, like a standard deviation of the SMA or FMA).

When processing circuitry 14 determines that an FMA varies from an SMA for a given physiological parameter by more than the associated difference threshold, processing circuitry 14 may determine that there is a risk that a MACE may occur to patient 4, for example, within a timeframe. In some examples, processing circuitry 14 may, based on the FMA varying from the SMA by more than the associated difference threshold, determine that such a risk of a MACE is significant. Being significant does not necessarily mean that the risk is greater than some percentage, but may mean that the risk is meaningful in view of potential patient outcomes if any underlying condition of patient 4 is not addressed.

In some examples, processing circuitry 14 may determine that there is a risk that a MACE may occur to patient 4, based on more than one respective FMA varying from a respective SMA by more than a respective difference threshold. For example, processing circuitry 14 may determine that a plurality of respective differences between respective FMAs and respective SMAs meet respective difference thresholds. Processing circuitry 50 may set respective flags indicative of each of the plurality of respective differences meeting the respective difference thresholds. Processing circuitry 50 may generate for output an indication of a risk that a MACE occurs based on at least two of the respective flags.

In response to, or based on, the determination that the FMA varies from the SMA for a given physiological parameter by more than the associated difference threshold, processing circuitry 14 may set a flag and/or generate an indication for output. The flag or the indication may be indicative of a risk of a MACE occurring to patient 4 within a timeframe, for example, within an amount of seconds, minutes, hours, days, weeks, or the like. For example, the flag or indication may be indicative of a risk of a MACE within a timeframe such as within x amount of days, y amount of hours, or between a and b amount of days or c and d amount of hours. In some examples, processing circuitry 14 may periodically generate flags (e.g., hourly or daily). Such flags may either indicate that a difference between a FMA and SMA for a given physiological parameter meets the associated difference threshold or does not meet the associated difference threshold. In some examples, processing circuitry 14 may output the indication so as to notify patient 4 and/or a clinician of the risk of the MACE occurring.

In some examples, processing circuitry 14 may determine, based on which physiological parameter or combination of physiological parameters may have an associated difference between an FMA and an SMA that is greater than (or greater than or equal to) an associated difference threshold for a respective physiological parameter, that a particular type(s) of issue(s) may cause the suspected MACE. Such type(s) of issues may include a structural issue, a coronary issue, and/or a conductive issue. For example, a structural issue may be a structural issue of a heart itself of patient 4. A coronary issue may be an issue of the vasculature nearby the heart of patient 4. A conductive issue may be an issue of the electrical physiology of patient 4. Physiological parameters which may be indicative of a possible MACE occurring, for example, within a timeframe, may include, but are not limited to, 1) glucose levels and derivatives, such as time in range; 2) respiration rate; 3) pulse oximeter parameters, such as SpO2 blood oxygen saturation and perfusion index; 4) heart rate and derivatives such as heart rate variability, and/or night heart rate; 5) blood pressure and derivatives, such as pulse pressure, mean and/or radial arterial pressure, central venous pressure; 6) physiological parameters discernable from acoustic signals, such as physiological parameters related to rate of blood flow and/or a pulse wave velocity; and/or 7) activity level.

For example, a high amount of atrial fibrillation may be indicative of conductive issue(s) and thus, conductive issues may be a cause of a MACE risk. A low amount of daily activity or low heart rate variability (HRV) may be indicative of a coronary issue which may be a cause of a MACE risk. Anomalies in blood flow (e.g., which may be determined by tracking heart sounds and determining any harmonics in the resulting waveforms that may not be normally present in a relatively normal structured anatomy and physiology) may be indicative of a structural issue which may be a cause of a MACE risk. In some examples, the flag and/or the indication may include an indication of which type of issue(s) patient 4 may be suffering from and/or which physiological parameter(s) may have an FMA that varies more from the SMA than the difference threshold. In some examples, the flag and/or the indication may include an indication of the difference between the FMA and the SMA and/or an indication of how much the FMA varies from the difference threshold.

External device 12 may be a hand-held computing device with a display viewable by the user and an interface for providing input to external device 12 (e.g., a user input mechanism). For example, external device 12 may include a display screen (e.g., a liquid crystal display (LCD) or a light emitting diode (LED) display) that presents information to the user. In addition, external device 12 may include a touch screen display, keypad, buttons, a peripheral pointing device, voice activation, or another input mechanism that allows the user to navigate through the user interface of external device 12 and provide input. If external device 12 includes buttons and a keypad, the buttons may be dedicated to performing a certain function, e.g., a power button, the buttons and the keypad may be soft keys that change in function depending upon the section of the user interface currently viewed by the user, or any combination thereof.

In some examples, external device 12 may be a separate application within another multi-function device, rather than a dedicated computing device. For example, the multi-function device may be a cellular phone, a tablet computer, a digital camera, or another computing device that may run an application that enables external device to operate as described herein.

When external device 12 is configured for use by the clinician, external device 12 may be used to transmit instructions to IMD 10 and/or wearable device 6, and to receive sensed signals, values of physiological parameters, flags, indications, or other information which may be sensed, processed, or determined by IMD 10 and/or wearable device 6. Example instructions may include requests to set electrode combinations for sensing and any other information that may be useful for programming into IMD 10 and/or wearable device 6. The clinician may also configure and store operational parameters for IMD 10 and/or wearable device 6 within IMD 10 and/or wearable device 6 with the aid of external device 12. In some examples, external device 12 assists the clinician in the configuration of IMD 10 and/or wearable device 6 by providing a system for identifying potentially beneficial operational parameter values.

Whether external device 12 is configured for clinician or patient use, external device 12 is configured to communicate with IMD 10 and/or wearable device 6, and, optionally, another computing device (not illustrated in FIG. 1), via wireless communication and/or wired or optical communication. External device 12, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies). In some examples, IMD 10 and wearable device 6 may be configured to communicate with each other via wireless communication.

Processing circuitry 14, in some examples, may include one or more processors that are configured to implement functionality and/or process instructions for execution within IMD 10, wearable device 6, and/or external device 12. For example, processing circuitry 14 may be capable of processing instructions stored in a storage device. Processing circuitry 14 may include, for example, microprocessors, digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 14 may include any suitable structure, whether in hardware, software, firmware, or any combination thereof, to perform the functions ascribed herein to processing circuitry 14.

Processing circuitry 14 may represent processing circuitry located within any combination of IMD 10, wearable device 6, and/or external device 12. In some examples, processing circuitry 14 may be entirely located within a housing of IMD 10. In other examples, processing circuitry 14 may be entirely located within or on wearable device 6. In other examples, processing circuitry 14 may be entirely located within a housing of external device 12. In other examples, processing circuitry 14 may be located within any combination of IMD 10, wearable device 6, external device 12, and another device or group of devices that are not illustrated in FIG. 1. As such, techniques and capabilities attributed herein to processing circuitry 14 may be attributed to any combination of IMD 10, wearable device 6, external device 12, and other devices that are not illustrated in FIG. 1.

Although in one example, IMD 10 takes the form of an ICM, in other examples, IMD 10 takes the form of any one or more of an ICM, a pacemaker, a defibrillator, a cardiac resynchronization therapy device, an implantable pulse generator, an intra-cardiac pressure measuring device, a ventricular assist device, a pulmonary artery pressure device, a subcutaneous blood pressure device, or the like. Although in one example, wearable device 6 takes the form of a patch, in some examples, wearable device 6 takes the form of any one or more of a pulse oximeter, a fitness tracker device, a watch, a wristband, a headband, a chest strap, a mask, a finger clip, ring, or the like. The physiological parameters discussed herein may be sensed or determined using one or more of the aforementioned devices, as well as external devices such as external device 12.

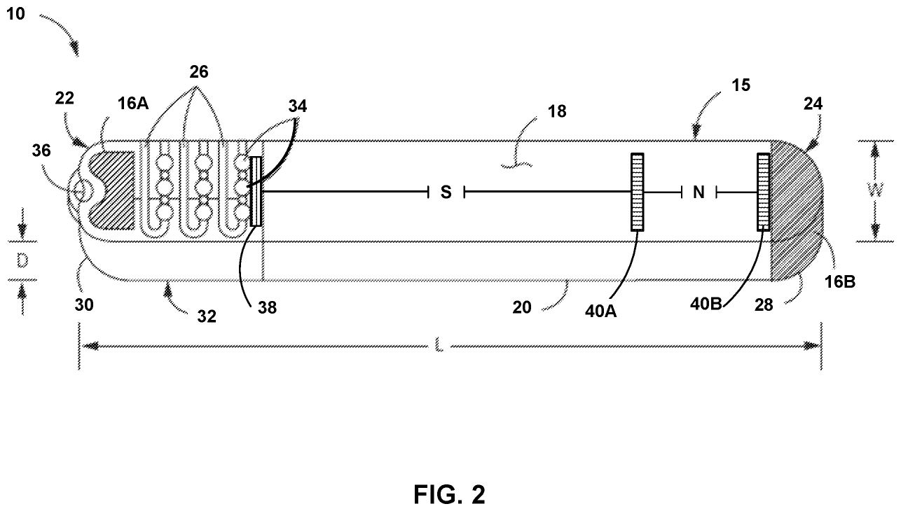

FIG. 2 is a conceptual drawing illustrating an example configuration of IMD 10 of the medical device system 2 of FIG. 1, in accordance with one or more techniques described herein. In the example shown in FIG. 2, IMD 10 may be a leadless, vascularly-implantable monitoring device having housing 15, proximal electrode 16A, and distal electrode 16B. Housing 15 may further include first major surface 18, second major surface 20, proximal end 22, and distal end 24. In some examples, IMD 10 may include one or more additional electrodes 16C, 16D positioned on one or both of major surfaces 18, 20 of IMD 10. Housing 15 encloses electronic circuitry located inside the IMD 10, and protects the circuitry contained therein from fluids such as body fluids (e.g., blood). In some examples, electrical feedthroughs provide electrical connection of electrodes 16A-16D, and antenna 26, to circuitry within housing 15. In some examples, electrode 16B may be formed from an uninsulated portion of conductive housing 15.

In the example shown in FIG. 2, IMD 10 is defined by a length L, a width W, and thickness or depth D. In this example, IMD 10 is in the form of an elongated rectangular prism in which length L is significantly greater than width W, and in which width W is greater than depth D. However, other configurations of IMD 10 are contemplated, such as those in which the relative proportions of length L, width W, and depth D vary from those described and shown in FIG. 2. In some examples, the geometry of the IMD 10, such as the width W being greater than the depth D, may be selected to allow IMD 10 to be inserted under the skin of the patient using a minimally invasive procedure and to remain in the desired orientation during insertion. In addition, IMD 10 may include radial asymmetries (e.g., the rectangular shape) along a longitudinal axis of IMD 10, which may help maintain the device in a desired orientation following implantation.

In some examples, a spacing between proximal electrode 16A and distal electrode 16B may range from about 30-55 mm, about 35-55 mm, or about 40-55 mm, or more generally from about 25-60 mm. Overall, IMD 10 may have a length L of about 20-30 mm, about 40-60 mm, or about 45-60 mm. In some examples, the width W of major surface 18 may range from about 3-10 mm, and may be any single width or range of widths between about 3-10 mm. In some examples, a depth D of IMD 10 may range from about 2-9 mm. In other examples, the depth D of IMD 10 may range from about 2-5 mm, and may be any single or range of depths from about 2-9 mm. In any such examples, IMD 10 is sufficiently compact to be implanted within the subcutaneous space of patient 4 in the region of a pectoral muscle.

IMD 10, according to an example of the present disclosure, may have a geometry and size designed for ease of implant and patient comfort. Examples of IMD 10 described in this disclosure may have a volume of 3 cubic centimeters (cm³) or less, 1.5 cm³ or less, or any volume therebetween. In addition, in the example shown in FIG. 2, proximal end 22 and distal end 24 are rounded to reduce discomfort and irritation to surrounding tissue once implanted under the skin of patient 4.

In the example shown in FIG. 2, first major surface 18 of IMD 10 faces outward towards the skin, when IMD 10 is inserted within patient 4, whereas second major surface 20 is faces inward toward musculature of patient 4. Thus, first and second major surfaces 18, 20 may face in directions along a sagittal axis of patient 4 (see FIG. 1), and this orientation may be generally maintained upon implantation due to the dimensions of IMD 10.

Proximal electrode 16A and distal electrode 16B may be used to sense cardiac electromyogram (EGM) signals (e.g., electrocardiogram (ECG) signals), sense impedances of tissue, or the like, when IMD 10 is implanted subcutaneously in patient 4. For example, IMD 10 may utilize signals sensed by proximal electrode 16A and distal electrode 16B to determine values of certain physiological parameters, such as respiration rate, heart rate, heart rate variability, night heart rate, or the like. Additionally, in some examples, electrodes 16A, 16B may be used by communication circuitry of IMD 10 for tissue conductance communication (TCC) communication with external device 12 or another device.

In the example shown in FIG. 2, proximal electrode 16A is in close proximity to proximal end 22, and distal electrode 16B is in close proximity to distal end 24 of IMD 10. In this example, distal electrode 16B is not limited to a flattened, outward facing surface, but may extend from first major surface 18, around rounded edges 28 or end surface 30, and onto the second major surface 20 in a three-dimensional curved configuration. As illustrated, proximal electrode 16A is located on first major surface 18 and is substantially flat and outward facing. However, in other examples not shown here, proximal electrode 16A and distal electrode 16B both may be configured like proximal electrode 16A shown in FIG. 2, or both may be configured like distal electrode 16B shown in FIG. 2. In some examples, additional electrodes 16C and 16D may be positioned on one or both of first major surface 18 and second major surface 20, such that a total of four electrodes are included on IMD 10. Any of electrodes 16A-16D may be formed of a biocompatible conductive material. For example, any of electrodes 16A-16D may be formed from any of stainless steel, titanium, platinum, iridium, or alloys thereof. In addition, electrodes of IMD 10 may be coated with a material such as titanium nitride or fractal titanium nitride, although other suitable materials and coatings for such electrodes may be used.

In the example shown in FIG. 2, proximal end 22 of IMD 10 includes header assembly 32 having one or more of proximal electrode 16A, integrated antenna 26, anti-migration projections 34, and suture hole 36. Integrated antenna 26 is located on the same major surface (e.g., first major surface 18) as proximal electrode 16A, and may be an integral part of header assembly 32. In other examples, integrated antenna 26 may be formed on the major surface opposite from proximal electrode 16A, or, in still other examples, may be incorporated within housing 15 of IMD 10. Antenna 26 may be configured to transmit or receive electromagnetic signals for communication. For example, antenna 26 may be configured to transmit to or receive signals from a programmer (e.g., external device 12) and/or wearable device 6 via inductive coupling, electromagnetic coupling, tissue conductance, Near Field Communication (NFC), Radio Frequency Identification (RFID), Bluetooth®, WiFi®, or other proprietary or non-proprietary wireless telemetry communication schemes. Antenna 26 may be coupled to communication circuitry of IMD 10, which may drive antenna 26 to transmit signals to external device 12 and/or wearable device 6, and may transmit signals received from external device 12 and/or wearable device 6 to processing circuitry of IMD 10 via communication circuitry.

In some examples, IMD 10 may include several features for retaining IMD 10 in position once subcutaneously implanted in patient 4, so as to decrease the chance that IMD 10 migrates in the body of patient 4. For example, as shown in FIG. 2, housing 15 may include anti-migration projections 34 positioned adjacent integrated antenna 26.

Anti-migration projections 34 may include a plurality of bumps or protrusions extending away from first major surface 18, and may help prevent longitudinal movement of IMD 10 after implantation in patient 4. In other examples, anti-migration projections 34 may be located on the opposite major surface as proximal electrode 16A and/or integrated antenna 26. In addition, in the example shown in FIG. 2 header assembly 32 includes suture hole 36, which provides another means of securing IMD 10 to the patient to prevent movement following insertion. In the example shown, suture hole 36 is located adjacent to proximal electrode 16A. In some examples, header assembly 32 may include a molded header assembly made from a polymeric or plastic material, which may be integrated or separable from the main portion of IMD 10.

In the example shown in FIG. 2, IMD 10 includes a light emitter 38, a proximal light detector 40A, and a distal light detector 40B positioned on housing 15 of IMD 10. Light detector 40A may be positioned at a distance S from light emitter 38, and a distal light detector 40B positioned at a distance S+N from light emitter 38. In other examples, IMD 10 may include only one of light detectors 40A, 40B, or may include additional light emitters and/or additional light detectors. Although light emitter 38 and light detectors 40A, 40B are described herein as being positioned on housing 15 of IMD 10, in other examples, one or more of light emitter 38 and light detectors 40A, 40B may be positioned, on a housing of another type of IMD within patient 4, such as a transvenous, subcutaneous, or extravascular pacemaker or ICD, or connected to such a device via a lead.

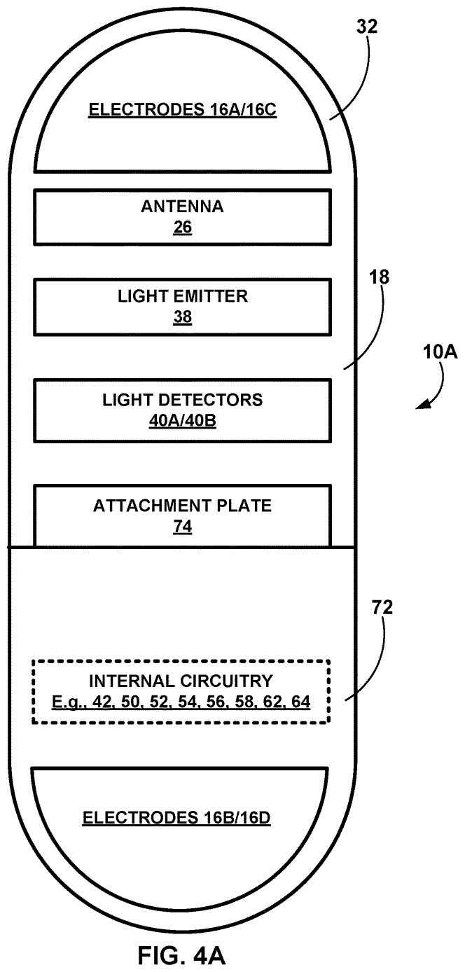

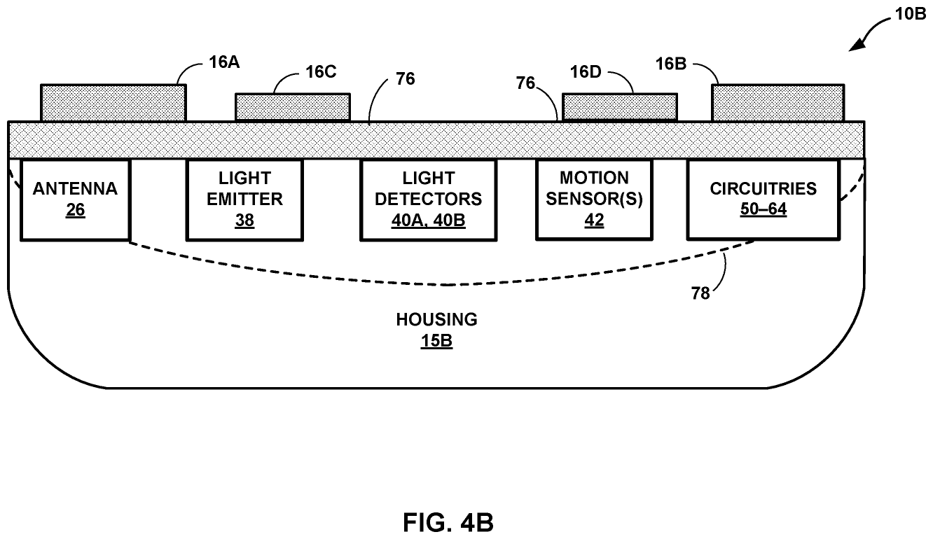

As shown in FIG. 2, light emitter 38 may be positioned on header assembly 32, although, in other examples, one or both of light detectors 40A, 40B may additionally or alternatively be positioned on header assembly 32. In some examples, light emitter 38 may be positioned on a medial section of IMD 10, such as part way between proximal end 22 and distal end 24. Although light emitter 38 and light detectors 40A, 40B are illustrated as being positioned on first major surface 18, light emitter 38, light detectors 40A, 40B alternatively may be positioned on second major surface 20. In some examples, IMD may be implanted such that light emitter 38 and light detectors 40A, 40B face inward when IMD 10 is implanted, toward the muscle of patient 4, which may help minimize interference from background light coming from outside the body of patient 4. Light detectors 40A, 40B may include a glass or sapphire window, such as described below with respect to FIG. 4B, or may be positioned beneath a portion of housing 15 of IMD 10 that is made of glass or sapphire, or otherwise transparent or translucent. In some examples, light detectors 40A, 40B may be configured to sense a signal indicative of Sp02 blood oxygen saturation and perfusion rate (e.g., pulse oximeter parameters), respiration rate, heart rate, heart rate variability, night heart rate, or the like.

In some examples, IMD 10 may include one or more additional sensors, such as one or more motion sensors, glucose sensors, acoustic sensors, pressure sensors, or the like (not shown in FIG. 2). For example, motion sensors may be 3D accelerometers configured to generate signals indicative of one or more types of movement of the patient, such as gross body movement (e.g., motion) of the patient, patient posture, movements associated with the beating of the heart, movements associated with respiration, or the movement of IMD 10 within the body of patient 4. Such signals may be used by IMD 10 to determine values of physiological parameters such as respiration rate, heart rate, heart rate variability, night heart rate (e.g., a lack of physical movement of patient 4 may be indicative of night time/sleep which may be used together with movements indicative of beating of the heart to determine night heart rate), patient activity levels, or the like. In some cases, One or more of the parameters monitored by IMD 10 (e.g., bio impedance, respiration rate, EGM, etc.) may fluctuate in response to changes in one or more such types of movement. For example, changes in parameter values sometimes may be attributable to increased patient motion (e.g., exercise or other physical motion as compared to immobility) or to changes in patient posture, and not necessarily to changes in a medical condition. Thus, in some techniques of identifying or tracking a medical condition of patient 4, it may be advantageous to account for such fluctuations when determining whether a change in a parameter is indicative of a change in a medical condition.

IMD 10 may determine values of physiological parameters such as glucose levels or associated physiological parameters, such as time in range, based on signals from one or more glucose sensors. In some examples, one or more glucose sensors may be disposed on an outer surface of IMD 10. IMD 10 may include acoustic sensors whose signals IMD 10 may utilize to determining values of physiological parameters such as respiration rate, heart rate, heart rate variability, night heart rate, and/or other physiological parameters discernable from acoustic signals, such as physiological parameters related to rate of blood flow and/or a pulse wave velocity. IMD 10 may include pressure sensors whose signals IMD 10 may use to determine respiration rate, blood pressure, and/or blood pressure derivatives, such as pulse pressure, mean and/or radial arterial pressure, central venous pressure, or the like.

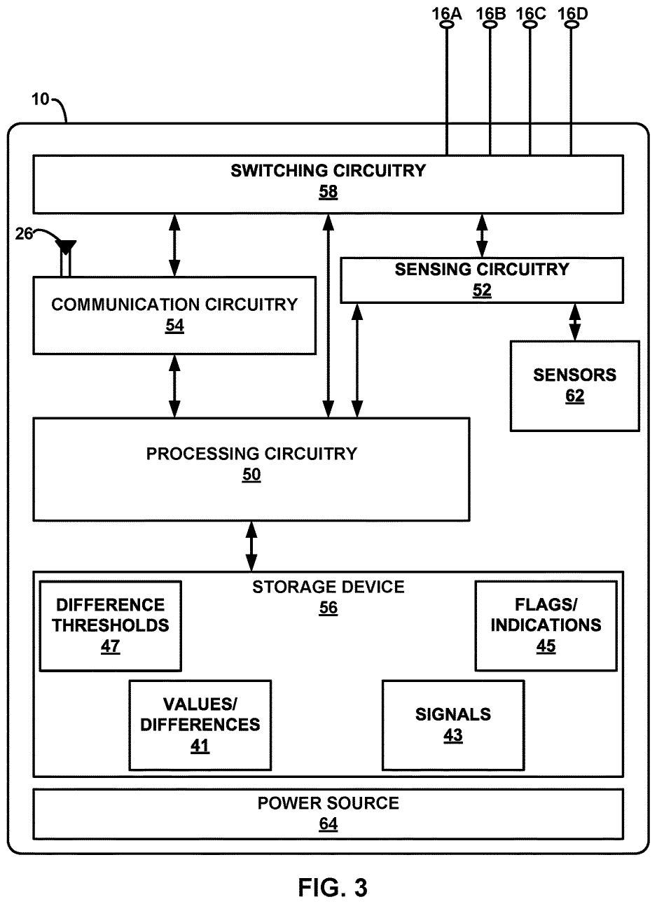

FIG. 3 is a functional block diagram illustrating an example configuration of IMD 10 of FIGS. 1 and 2, in accordance with one or more techniques described herein. In the illustrated example, IMD 10 includes electrodes 16, antenna 26, processing circuitry 50, sensing circuitry 52, communication circuitry 54, storage device 56, switching circuitry 58, sensors 62 including motion sensor(s) 42 (which may include an accelerometer), and power source 64. In some examples, FIG. 3 may depict an example configuration of wearable device 6. It should be noted that, in some examples, IMD 10 and/or wearable device 6 may include fewer or more components than are depicted in FIG. 3.

Processing circuitry 50 may include fixed function circuitry and/or programmable processing circuitry. Processing circuitry 50 may include any one or more of a microprocessor, a controller, a DSP, an ASIC, an FPGA, or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof. In some examples, one or more techniques of this disclosure may be performed by processing circuitry 50.

Sensing circuitry 52 and communication circuitry 54 may be selectively coupled to electrodes 16A-16D via switching circuitry 58, as controlled by processing circuitry 50. Sensing circuitry 52 may monitor signals from electrodes 16A-16D in order to monitor, for example, electrical activity of heart. Sensing circuitry 52 also may monitor signals from sensors 62, which may include motion sensor(s) (which may include an accelerometer), glucose sensor(s), acoustic sensor(s), light sensor(s), pressure sensor(s), or the like. In some examples, sensing circuitry 52 may include one or more filters and amplifiers for filtering and amplifying signals received from one or more of electrodes 16A-16D and/or sensors 62. Such signals may be indicative of physiological parameters of patient 4. Processing circuitry 50 may process such signals to determine values of various physiological parameters. For example, processing circuitry 50 may monitor signal(s) from glucose sensor(s) of sensors 62 to determine values of glucose levels and derivatives, such as time in range. For example, processing circuitry 50 may monitor signal(s) from electrodes 16A-16D, light sensor(s) of sensors 62, acoustic sensor(s) of sensors 62 and/or pressure sensor(s) of sensors 62 to determine values of respiration rate. Processing circuitry 50 may monitor signal(s) from light sensor(s) of sensors 62 to determine values of pulse oximeter parameters, such as SpO2 blood oxygen saturation and perfusion index. Processing circuitry 50 may monitor signal(s) from electrodes 16A-16D, light sensor(s) of sensors 62 and/or acoustic sensor(s) of sensors 62 to determine values of heart rate and derivatives such as heart rate variability, and/or night heart rate.

Processing circuitry 50 may monitor signal(s) from pressure sensor(s) of sensors 62 to determine values of blood pressure and derivatives, such as pulse pressure, mean and/or radial arterial pressure, central venous pressure. Processing circuitry 50 may monitor signal(s) of acoustic sensor(s) of sensors 62 to determine values of physiological parameters discernable from acoustic signals, such as physiological parameters related to rate of blood flow and/or a pulse wave velocity.

Processing circuitry 50 may obtain one or more signals indicative of one or more respective physiological parameters, for example, from electrodes 16A-16D, sensing circuitry 52, and/or sensors 62. Processing circuitry 50 may determine respective values of the one or more respective physiological parameters based on the one or more signals. Processing circuitry 50 may determine a respective SMA of a first subset of the respective values of the one or more respective physiological parameters and a respective FMA of a second subset of the respective values of the one or more respective physiological parameters. Processing circuitry 50 may determine that a respective difference between the respective FMA and the respective SMA meets a respective difference threshold. Processing circuitry 50 may, based on the respective difference meeting the respective difference threshold, at least one of (i) set a flag indicative a risk that a major adverse cardiac event (MACE) occurs or (ii) generate for output an indication of a risk that a MACE occurs.

Such an indication may include an alert that a MACE may occur, such as a MACE may occur within a timeframe. The indication may further include an estimated percentage of risk that the MACE may occur within the timeframe. In some examples, the flag or indication may include a type of issue that may cause the alert, such as a structural, coronary, and/or conductive issue.

In some examples, the indication may include instructions to patient 4 to seek medical help or to make an appointment with a clinician, and/or instructions to a clinician on which test(s) the clinician should consider performing on patient 4. In some examples, the indication may include one or more of value(s) of the physiological parameter(s), the SMA(s), the FMA(s), the difference threshold(s), the variance(s) between an SMA and an FMA, the variance(s) between a difference between an SMA and FMA and an associated difference threshold, or the like. In some examples, processing circuitry 50 may control communication circuitry 54 to output flags/indications 45 (including the indication) to transmit flags/indications 45 to external device 12, where processing circuitry of external device 12 may display or otherwise present the indication to patient 4 and/or a clinician via a user interface.

Communication circuitry 54 may include any suitable hardware, firmware, software or any combination thereof for communicating with another device, such as external device 12 or another IMD or sensor, such as a pressure sensing device. Under the control of processing circuitry 50, communication circuitry 54 may receive downlink telemetry from, as well as send uplink telemetry to, external device 12 or another device (e.g., wearable device 6) with the aid of an internal or external antenna, e.g., antenna 26 (FIG. 2). In addition, processing circuitry 50 may communicate with a networked computing device via an external device (e.g., external device 12) and a computer network, such as the Medtronic CareLink® Network developed by Medtronic, plc, of Dublin, Ireland.

A clinician or other user may retrieve data from IMD 10 using external device 12, or by using another local or networked computing device configured to communicate with processing circuitry 50 via communication circuitry 54. The clinician may also program parameters of IMD 10 using external device 12 or another local or networked computing device.

In some examples, storage device 56 includes computer-readable instructions that, when executed by processing circuitry 50, cause IMD 10 and processing circuitry 50 to perform various functions attributed to IMD 10 and processing circuitry 50 herein.

Storage device 56 may include any volatile, non-volatile, magnetic, optical, or electrical media, such as a random access memory (RAM), ferroelectric RAM (FRAM) read-only memory (ROM), non-volatile RAM (NVRAM), electrically-erasable programmable ROM (EEPROM), flash memory, or any other digital media.

Storage device 56 may also store difference thresholds 47. In some examples, difference thresholds 47 may include a plurality of respective difference thresholds-one corresponding to each physiological parameter which may be monitored to determine whether there is a risk of a MACE occurring to patient 4. In some examples, the difference thresholds are programmable, for example, by a clinician. In some examples, the difference thresholds include absolute values and are not derived by processing circuitry 50 based on other information, such as a SMA or an FMA.

Storage device 56 may also store determined values and/or differences, e.g., in values/differences 41. For example, storage device 56 may store respective physiological parameter values in values/differences 41. Storage device 56 may, additionally, or alternatively, store determined differences between respective SMAs and respective FMAs and/or respective differences between the determined differences between respective SMAs and respective FMAs and respective difference thresholds (of difference thresholds 47).

Storage device 56 may also store signals 43. For example, any of, or each of, the signals indicative of physiological parameters (or portions thereof) may be stored in signals 43, for example, for transmission to, or retrieval by, external device 12.

Storage device 56 may also store flags/indications 45. For example, any generated flags or indications may be stored in flags/indications 45 for transmission to, or retrieval by, external device 12.

Power source 64 is configured to deliver operating power to the components of IMD 10. Power source 64 may include a battery and a power generation circuit to produce the operating power. In some examples, the battery is rechargeable to allow extended operation. In some examples, recharging is accomplished through proximal inductive interaction between an external charger and an inductive charging coil within external device 12. Power source 64 may include any one or more of a plurality of different battery types, such as nickel cadmium batteries and lithium-ion batteries. A non-rechargeable battery may be selected to last for several years, while a rechargeable battery may be inductively charged from an external device, e.g., on a daily or weekly basis.

While not described with respect to a separate FIG., wearable device 6 may include one or more similar components to those of IMD 10 of FIG. 3. It should be noted that some types of sensors may be implementable in wearable device 6 that may not be practical in IMD 10. For example, wearable device 6 may include a girth sensor (e.g., when wearable device 6 includes a chest strap) or a flow meter (e.g., when wearable device 6 includes a mask) which may sense a signal indicative of respiration rate.

FIGS. 4A and 4B illustrate two additional example IMDs that may be substantially similar to IMD 10 of FIGS. 1-3, but which may include one or more additional features, in accordance with one or more techniques described herein. The components of FIGS. 4A and 4B may not necessarily be drawn to scale, but instead may be enlarged to show detail. FIG. 4A is a block diagram of a top view of an example configuration of an IMD 10A. FIG. 4B is a block diagram of a side view of example IMD 10B, which may include an insulative layer as described below.

FIG. 4A is a conceptual drawing illustrating another example IMD 10A that may be substantially similar to IMD 10 of FIG. 1. In addition to the components illustrated in FIGS. 1-3, the example of IMD 10 illustrated in FIG. 4A also may include a body portion 72 and an attachment plate 74. Attachment plate 74 may be configured to mechanically couple header assembly 32 to body portion 72 of IMD 10A. Body portion 72 of IMD 10A may be configured to house one or more of the internal components of IMD 10 illustrated in FIG. 3, such as one or more of processing circuitry 50, sensing circuitry 52, communication circuitry 54, storage device 56, switching circuitry 58, internal components of sensors 62, and power source 64. In some examples, body portion 72 may be formed of one or more of titanium, ceramic, or any other suitable biocompatible materials.

FIG. 4B is a conceptual drawing illustrating another example IMD 10B that may include components substantially similar to IMD 10 of FIG. 1. In addition to the components illustrated in FIGS. 1-3, the example of IMD 10B illustrated in FIG. 4B also may include a wafer-scale insulative cover 76, which may help insulate electrical signals passing between electrodes 16A-16D, light detectors 40A, 40B on housing 15B and processing circuitry 50. In some examples, insulative cover 76 may be positioned over an open housing 15 to form the housing for the components of IMD 10B. One or more components of IMD 10B (e.g., antenna 26, light emitter 38, light detectors 40A, 40B, processing circuitry 50, sensing circuitry 52, communication circuitry 54, switching circuitry 58, and/or power source 64) may be formed on a bottom side of insulative cover 76, such as by using flip-chip technology. Insulative cover 76 may be flipped onto a housing 15B. When flipped and placed onto housing 15B, the components of IMD 10B formed on the bottom side of insulative cover 76 may be positioned in a gap 78 defined by housing 15B.

Insulative cover 76 may be configured so as not to interfere with the operation of IMD 10B. For example, one or more of electrodes 16A-16D may be formed or placed above or on top of insulative cover 76, and electrically connected to switching circuitry 58 through one or more vias (not shown) formed through insulative cover 76. Insulative cover 76 may be formed of sapphire (e.g., corundum), glass, parylene, and/or any other suitable insulating material. Sapphire may be greater than 80% transmissive for wavelengths in the range of about 300 nm to about 4000 nm, and may have a relatively flat profile. In the case of variation, different transmissions at different wavelengths may be compensated for, such as by using a ratiometric approach. In some examples, insulative cover 76 may have a thickness of about 300 micrometers to about 600 micrometers. Housing 15B may be formed from titanium or any other suitable material (e.g., a biocompatible material), and may have a thickness of about 200 micrometers to about 500 micrometers. These materials and dimensions are examples only, and other materials and other thicknesses are possible for devices of this disclosure.

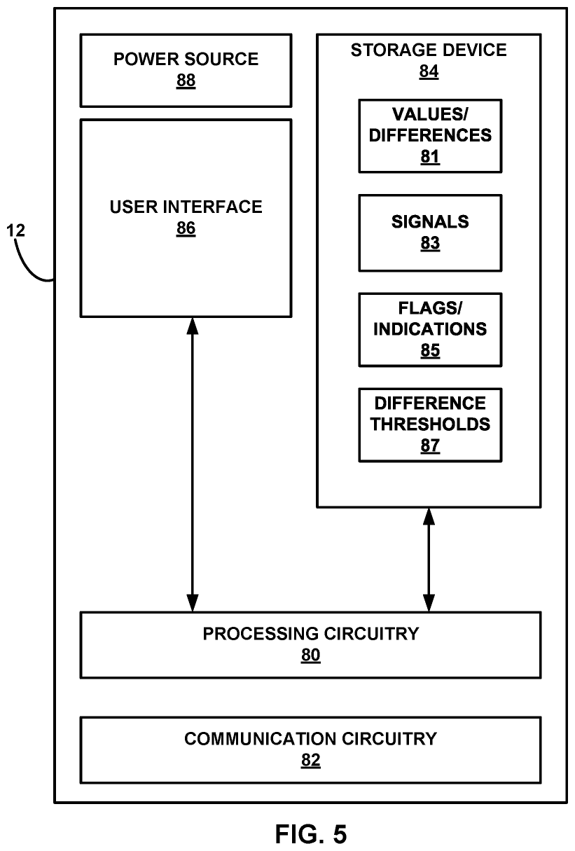

FIG. 5 is a block diagram illustrating an example configuration of components of external device 12, in accordance with one or more techniques of this disclosure. In the example of FIG. 5, external device 12 includes processing circuitry 80, communication circuitry 82, storage device 84, user interface 86, and power source 88. In some examples, external device 12 may include additional components not depicted in FIG. 5 or fewer components than depicted in FIG. 5.

Processing circuitry 80, in one example, may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80. In some examples, processing circuitry 80 may perform one or more of the techniques of this disclosure.

Processing circuitry 80 may receive a flag and/or indication from IMD 10 and/or wearable device 6 indicative of a risk of a MACE occurring within a timeframe.

In some examples, for example, when processing circuitry 80 receives such a flag, processing circuitry 80 may generate an indication for output. Such an indication may include an alert that a MACE may occur within a timeframe. The indication may further include an estimated percentage of risk that the MACE may occur within the timeframe. In some examples, the flag or indication may include a type of issue that may cause the alert, such as a structural, coronary, and/or conductive issue. For example, processing circuitry 80 may control user interface 86 to display or otherwise present the indication to patient 4 and/or a clinician.

In some examples, rather than receive the flag and/or indication from IMD 10 and/or wearable device 6, processing circuitry 80 may obtain values of the one or more respective physiological parameters and determine the respective SMA of the first subset of the respective values of the one or more respective physiological parameters and the respective FMA of the second subset of the respective values of the one or more respective physiological parameters. Processing circuitry 80 may determine that the respective difference between the respective FMA and the respective SMA meets the respective difference threshold. In some examples, processing circuitry 80 may, based on the respective difference meeting the respective difference threshold, at least one of (i) set a flag indicative a risk that a major adverse cardiac event (MACE) occurs or (ii) generate for output an indication of a risk that a MACE occurs. In some examples, rather than determine the respective SMA of the first subset of the respective values of the one or more respective physiological parameters and the respective FMA of the second subset of the respective values of the one or more respective physiological parameters, processing circuitry 80 may obtain the respective SMA and the respective FMA from IMD 10.

In some examples, the indication may include instructions to patient 4 to seek medical help or to make an appointment with a clinician, and/or instructions to a clinician on which test(s) the clinician should consider performing on patient 4. In some examples, the indication may include one or more of value(s) of the physiological parameter(s), the SMA(s), the FMA(s), the difference threshold(s), the variance(s) between an SMA and an FMA, the variance(s) between a difference between an SMA and FMA and an associated difference threshold, or the like.

Communication circuitry 82 may include any suitable hardware, firmware, software or any combination thereof for communicating with another device, such as IMD 10 and/or wearable device 6. Under the control of processing circuitry 80, communication circuitry 82 may receive downlink telemetry from, as well as send uplink telemetry to, IMD 10, or another device, such as wearable device 6. For example, communication circuitry 82 may receive from IMD 10 and/or wearable device 6 a flag and/or alert regarding a risk of a MACE.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, dynamic random access memories (DRAM), static random access memories (SRAM), magnetic discs, optical discs, flash memories, or forms of electrically programmable memories (EPROM) or EEPROM. In some examples, storage device 84 is used to store data indicative of instructions for execution by processing circuitry 80. Storage device 84 may be used by software or applications running on external device 12 to temporarily store information during program execution.

Storage device 84 may also store information which external device 12 may receive from IMD 10. For example, communication circuitry 82 may receive information from IMD 10 and processing circuitry 80 may store that information in storage device 84.

For example, Storage device 84 may store difference thresholds 87, which may correspond to difference thresholds 47 of FIG. 3. In this manner, a user of external device 12 may be able to view one or more of difference thresholds 47, for example, via user interface 86 and program one or more of the difference thresholds for upload to difference thresholds 47 of IMD 10, for example.

Storage device 84 may also store values/differences 81, which may correspond to values/differences 41 of FIG. 3. Storage device 84 may also store signals 83, which may correspond to signals 43 of FIG. 3. Storage device 56 may also store flags/indications 85, which may correspond to flags/indications 45.

Data exchanged between external device 12, IMD 10, and/or wearable device 6 may include operational parameters. External device 12 may transmit data including computer readable instructions which, when implemented by IMD 10 and/or wearable device 6, may control IMD 10 and/or wearable device 6 to change one or more operational parameters and/or export collected data. For example, processing circuitry 80 may transmit an instruction to IMD 10 which requests IMD 10 to export collected data (e.g., data corresponding to sensed physiological parameters, SMAs, FMAs, comparisons (including differences and/or variances), flags, indications, a suspected type of issue leading to a risk of MACE or other data discussed herein). In turn, external device 12 may receive the collected data from IMD 10 and store the collected data in storage device 84. Additionally, or alternatively, processing circuitry 80 may export instructions to IMD 10 and/or wearable device 6 requesting IMD 10 and/or wearable device 6 to update one or more operational parameters of IMD 10 and/or wearable device 6.

A user, such as a clinician, patient 4, or a caregiver, may interact with external device 12 through user interface 86. User interface 86 includes a display (not shown), such as an LCD or LED display or other type of screen, with which processing circuitry 80 may present information related to IMD 10 and/or wearable device 6 (e.g., generated indications). In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input.

Power source 88 is configured to deliver operating power to the components of external device 12. Power source 88 may include a battery and a power generation circuit to produce the operating power. In some examples, the battery is rechargeable to allow extended operation. Recharging may be accomplished by electrically coupling power source 88 to a cradle or plug that is connected to an alternating current (AC) outlet. In addition, recharging may be accomplished through proximal inductive interaction between an external charger and an inductive charging coil within external device 12. In other examples, traditional batteries (e.g., nickel cadmium or lithium-ion batteries) may be used. In addition, external device 12 may be directly coupled to an alternating current outlet to operate.

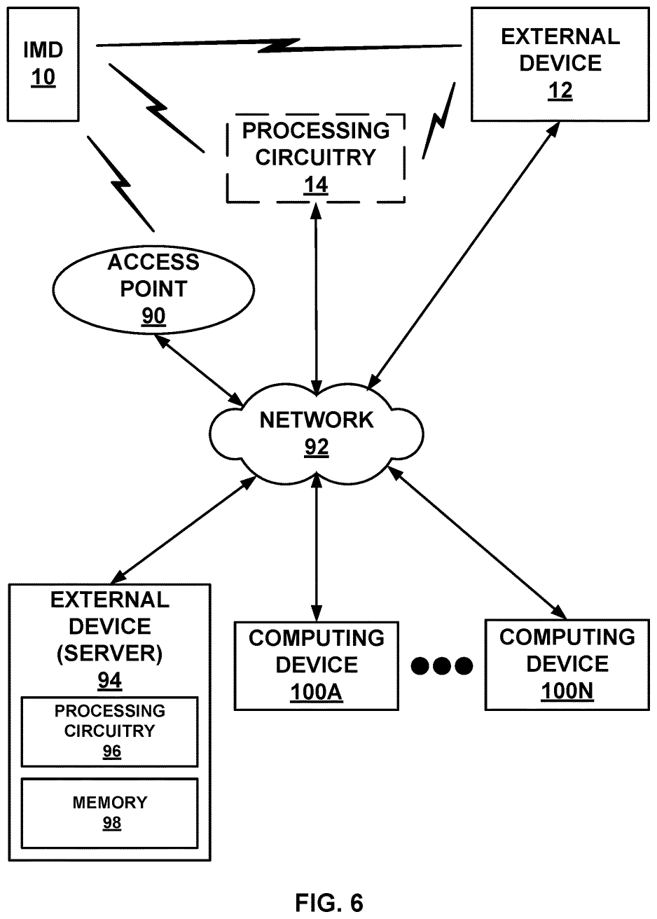

FIG. 6 is a block diagram illustrating an example system that includes an access point 90, a network 92, external computing devices, such as a server 94, and one or more other computing devices 100A-100N, which may be coupled to IMD 10, wearable device 6, external device 12, and/or processing circuitry 14 via network 92, in accordance with one or more techniques described herein. While not shown in FIG. 6, wearable device 6 may function similarly to IMD 10 as described with respect to FIG. 6 in systems including wearable device 6. In this example, IMD 10 may use communication circuitry 54 to communicate with external device 12 via a first wireless connection, and to communication with an access point 90 via a second wireless connection. In the example of FIG. 6, access point 90, external device 12, server 94, and computing devices 100A-100N are interconnected and may communicate with each other through network 92.

Access point 90 may include a device that connects to network 92 via any of a variety of connections, such as telephone dial-up, digital subscriber line (DSL), fiber optic, or cable modem connections. In other examples, access point 90 may be coupled to network 92 through different forms of connections, including wired or wireless connections. In some examples, access point 90 may be a user device, such as a tablet or smartphone, that may be co-located with the patient. As discussed above, IMD 10 may be configured to transmit data, such as values/differences 41, signals 43, and/or flags/indications 45, or other data collected by IMD 10 to external device 12. In addition, access point 90 may interrogate IMD 10, such as periodically or in response to a command from the patient or network 92, in order to retrieve information, such as physiological parameter values determined by processing circuitry 50 of IMD 10, or other operational or patient data from IMD 10. Access point 90 may then communicate the retrieved data to server 94 via network 92.

In some cases, server 94 may be configured to provide a secure storage site for data that has been collected from IMD 10, and/or external device 12, such as values/differences 41, signals 43, and/or flags/indications 45 and/or other information relating to patient 4. In some cases, server 94 may assemble data in web pages or other documents for viewing by trained professionals, such as clinicians, via computing devices 100A-100N. One or more aspects of the illustrated system of FIG. 6 may be implemented with general network technology and functionality, which may be similar to that provided by the Medtronic CareLink® Network developed by Medtronic plc, of Dublin, Ireland.

Server 94 may include processing circuitry 96. Processing circuitry 96 may include fixed function circuitry and/or programmable processing circuitry. Processing circuitry 96 may include any one or more of a microprocessor, a controller, a DSP, an ASIC, an FPGA, or equivalent discrete or analog logic circuitry. In some examples, processing circuitry 96 may include multiple components, such as any combination of one or more microprocessors, one or more controllers, one or more DSPs, one or more ASICs, or one or more FPGAs, as well as other discrete or integrated logic circuitry. The functions attributed to processing circuitry 96 herein may be embodied as software, firmware, hardware or any combination thereof. In some examples, processing circuitry 96 may perform one or more techniques described herein.

Server 94 may include memory 98. Memory 98 includes computer-readable instructions that, when executed by processing circuitry 96, cause IMD 10 and processing circuitry 96 to perform various functions attributed to IMD 10 and processing circuitry 96 herein. Memory 98 may include any volatile, non-volatile, magnetic, optical, or electrical media, such as RAM, ROM, NVRAM, EEPROM, flash memory, or any other digital media.

In some examples, one or more of computing devices 100A-100N (e.g., device 100A) may be a tablet or other smart device located with a clinician, by which the clinician may program, receive alerts from, and/or interrogate IMD 10 and/or external device 12. For example, the clinician may receive an indication regarding a risk of a MACE occurring to patient 4 and/or access values/differences 41, signals 43, flags/indications 45, difference thresholds 47, or the like, through device 100A, such as when patient 4 is in between clinician visits or when IMD 10 determines a risk of a MACE. In some examples, the clinician may enter instructions for a medical intervention for patient 4 into an app in device 100A, such as based on an indication for output and/or data associate with the indication, and/or based on other patient data known to the clinician. Device 100A then may transmit the instructions for medical intervention to another of computing devices 100A-100N (e.g., device 100B) located with patient 4 or a caregiver of patient 4. For example, such instructions for medical intervention may include an instruction to change a drug dosage, timing, or selection, to schedule a visit with the clinician, to take their fluid medication, or to seek medical attention. In further examples, device 100B may output an indication to patient 4, such as an alert to patient 4 based on the risk of a MACE occurring, which may enable patient 4 proactively to seek medical attention prior to receiving instructions for a medical intervention. In this manner, patient 4 may be empowered to take action, as needed, to address their medical status, which may help improve clinical outcomes for patient 4.

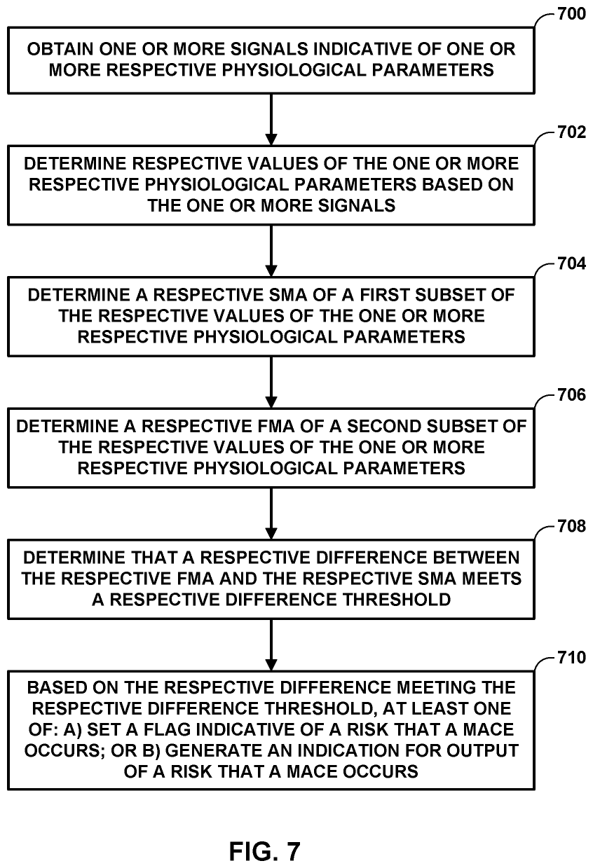

FIG. 7 is a flow diagram illustrating example MACE prediction techniques in accordance with one or more aspects of this disclosure. While discussed herein with respect to IMD 10 and processing circuitry 50 of IMD 10, it should be noted that the techniques of FIG. 7 may be performed by any device or combination of devices described herein which are capable of performing such techniques. For example, processing circuitry 14 may perform the techniques ascribed herein to processing circuitry 50.

Processing circuitry 50 may obtain one or more signals indicative of one or more respective physiological parameters (700). For example, processing circuitry 50 may receive from sensing circuitry 52 one or more signals indicative of one or more respective physiological parameters. Processing circuitry 50 may determine respective values of the one or more respective physiological parameters based on the one or more signals (702). For example, processing circuitry 50 may determine, based on a signal received from sensing circuitry 52, values of a physiological parameter over time.

Processing circuitry 50 may determine a respective SMA of a first subset of the respective values of the one or more respective physiological parameters (704). For example, processing circuitry 50 may average values of a physiological parameter sampled over a first, relatively longer time period to determine the respective SMA.

Processing circuitry 50 may determine a respective FMA of a second subset of the respective values of the one or more respective physiological parameters (706). For example, processing circuitry 50 may average values of a physiological parameter sampled over a second, relatively shorter time period. For example, the SMA period may be longer than the FMA period and the first subset of values may include the second subset of values. In some examples, the samples used to determine the SMA may include the samples used to determine the FMA plus additional samples, as the SMA includes samples over a longer time period. As an example, the SMA may be determined based on 100 samples of values of a physiological parameter and the FMA may be determined based on 10 samples of values of the physiological parameter and the 10 samples used to determine the FMA are among the 100 samples used to determine the SMA.

Processing circuitry 50 may determine that a respective difference between the respective FMA and the respective SMA meets a respective difference threshold (708). For example, processing circuitry 50 may determine a difference between an FMA for a given physiological parameter and the corresponding SMA. Processing circuitry 50 may determine whether that difference between the FMA and the SMA meets the difference threshold. For example, the difference may meet the difference threshold if the difference is greater than the difference threshold, or in other examples, if the difference is greater than or equal to the difference threshold. In some examples, each of the one or more respective difference thresholds is programmable. For example, a clinician may program any of the respective difference thresholds, for example, based on medical history of patient 4, to tune the time period within which the MACE may occur, or the like.

Processing circuitry 50, based on the respective difference meeting the respective difference threshold, at least one of set a flag indicative of a risk that a MACE occurs or generate an indication for output of a risk that a MACE occurs (710). For example, processing circuitry 50 may generate a flag and/or an indication indicative of a risk that a MACE occurs to patient 4 within a timeframe. The indication may include an alert that a MACE may occur, such as a MACE may occur within a timeframe. The indication may further include an estimated percentage of risk that the MACE may occur within the timeframe. In some examples, the flag or indication may include a type of issue that may cause the alert, such as a structural, coronary, and/or conductive issue.

In some examples, the indication may include instructions to patient 4 to seek medical help or to make an appointment with a clinician, and/or instructions to a clinician on which test(s) the clinician should consider performing on patient 4. In some examples, the indication may include one or more of value(s) of the physiological parameter(s), the SMA(s), the FMA(s), the difference threshold(s), the variance(s) between an SMA and an FMA, the variance(s) between a difference between an SMA and FMA and an associated difference threshold, or the like.

In some examples, processing circuitry 50 may determine that a plurality of respective differences between respective FMAs and respective SMAs meet respective difference thresholds. Processing circuitry 50 may set respective flags indicative of each of the plurality of respective differences meeting the respective difference thresholds. Processing circuitry 50 may generate for output an indication of a risk that a MACE occurs based on at least two of the respective flags. For example, processing circuitry 50 may determine that a risk that a MACE occurs based on more than one respective difference meeting respective difference thresholds.

In some examples, the one or more respective physiological parameters include at least one of a glucose level, a glucose time in range, a respiration rate, SpO2 blood oxygen saturation, blood oxygen perfusion index, heart rate, heart rate variability, night heart rate, blood pressure, pulse pressure, mean arterial pressure, radial arterial pressure, central venous pressure, pulse wave velocity or activity level.

In some examples, the system includes one or more respective sensors, the one or more respective sensors being configured to sense the one or more respective physiological parameters. In some examples, the one or more respective sensors include at least one of an implantable sensor or a wearable sensor.

In some examples, processing circuitry 50 is further configured to determine a type of issue associated with the risk that the MACE occurs based on the respective difference meeting the respective difference threshold, wherein the indication of the risk that the MACE occurs comprises an indication of the type of issue. In some examples, the type of issue includes at least one of a coronary issue, a structural issue, or a conductive issue.

In some examples, the indication includes a timeframe of the risk of the MACE. In some examples, the respective SMA includes a mean, median, or mode of the respective physiological parameter over an SMA period, the SMA period being a range measured in seconds, hours, days, months, or years. In some examples, the respective FMA includes a mean, median, or mode of the respective physiological parameter over an FMA period, the FMA period being a range measured in seconds, hours, days, months, or years. In some examples, the SMA is determined over an SMA period and the FMA is determined over an FMA period, and wherein the FMA period is shorter than the SMA period. In some examples, the SMA period is longer than the FMA period.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as clinician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, FRAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

This disclosure includes the following non-limiting examples.

Example 1. A system comprising: a memory configured to store physiological parameters of a patient; and processing circuitry communicatively coupled to the memory, the processing circuitry being configured to: obtain one or more signals indicative of one or more respective physiological parameters; determine respective values of the one or more respective physiological parameters based on the one or more signals; determine a respective slow-moving average (SMA) of a first subset of the respective values of the one or more respective physiological parameters; determine a respective fast-moving average (FMA) of a second subset of the respective values of the one or more respective physiological parameters; determine that a respective difference between the respective FMA and the respective SMA meets a respective difference threshold; and based on the respective difference meeting the respective difference threshold, at least one of: a) set a flag indicative a risk that a major adverse cardiac event (MACE) occurs; or b) generate for output an indication of a risk that a MACE occurs.

Example 2. The system of example 1, wherein the processing circuitry is further configured to: determine that a plurality of respective differences between respective FMAs and respective SMAs meet respective difference thresholds; set respective flags indicative of each of the plurality of respective differences meeting the respective difference thresholds; and generate for output an indication of a risk that a MACE occurs based on at least two of the respective flags.

Example 3. The system of example 1 or example 2, wherein the one or more respective physiological parameters comprise at least one of a glucose level, a glucose time in range, a respiration rate, SpO2 blood oxygen saturation, blood oxygen perfusion index, heart rate, heart rate variability, night heart rate, blood pressure, pulse pressure, mean arterial pressure, radial arterial pressure, central venous pressure, pulse wave velocity or activity level.

Example 4. The system of any of examples 1-3, further comprising one or more respective sensors, the one or more respective sensors being configured to sense the one or more respective physiological parameters.

Example 5. The system of example 4, wherein the one or more respective sensors comprise at least one of an implantable sensor or a wearable sensor.

Example 6. The system of any of examples 1-5, wherein the processing circuitry is further configured to determine a type of issue associated with the risk that the MACE occurs based on the respective difference meeting the respective difference threshold, wherein the indication of the risk that the MACE occurs comprises an indication of the type of issue.

Example 7. The system of example 6, wherein the type of issue includes at least one of a coronary issue, a structural issue, or a conductive issue.

Example 8. The system of any of examples 1-7, wherein the indication comprises a timeframe of the risk of the MACE.

Example 9. The system of any of examples 1-8, wherein the respective SMA comprises a mean, median, or mode of the respective physiological parameter over an SMA period, the SMA period being a range measured in seconds, hours, days, months, or years.

Example 10. The system of any of examples 1-9, wherein the respective FMA comprises a mean, median, or mode of the respective physiological parameter over an FMA period, the FMA period being a range measured in seconds, hours, days, months, or years.

Example 11. The system of any of claims 1-10, wherein the SMA is determined over an SMA period and the FMA is determined over an FMA period, and wherein the FMA period is shorter than the SMA period.

Example 12. A method comprising: obtaining, by processing circuitry, one or more signals indicative of one or more respective physiological parameters; determining, by the processing circuitry, respective values of the one or more respective physiological parameters based on the one or more signals; determining, by the processing circuitry, a respective slow-moving average (SMA) of a first subset of the respective values of the one or more respective physiological parameters; determining, by the processing circuitry, a respective fast-moving average (FMA) of a second subset of the respective values of the one or more respective physiological parameters; determining, by the processing circuitry, that a respective difference between the respective FMA and the respective SMA meets a respective difference threshold; and based on the respective difference meeting the respective difference threshold, at least one of: a) setting by the processing circuitry, a flag indicative a risk that a major adverse cardiac event (MACE) occurs; or b) generating by the processing circuitry, for output an indication of a risk that a MACE occurs.

Example 13. The method of example 12, further comprising: determining, by the processing circuitry, that a plurality of respective differences between respective FMAs and respective SMAs meet respective difference thresholds; setting, by the processing circuitry, respective flags indicative of each of the plurality of respective differences meeting the respective difference thresholds; and generating, by the processing circuitry, for output an indication of a risk that a MACE occurs based on at least two of the respective flags.
Example 14. The method of example 12 or example 13, wherein the one or more respective physiological parameters comprise at least one of a glucose level, a glucose time in range, a respiration rate, SpO2 blood oxygen saturation, blood oxygen perfusion index, heart rate, heart rate variability, night heart rate, blood pressure, pulse pressure, mean arterial pressure, radial arterial pressure, central venous pressure, pulse wave velocity, or activity level.

Example 15. The method of any of examples 12-14, further comprising sensing, by one or more respective sensors, the one or more respective physiological parameters.

Example 16. The method of example 15, wherein the one or more respective sensors comprise at least one of an implantable sensor or a wearable sensor.

Example 17. The method of any of examples 12-16, further comprising determining, by the processing circuitry, a type of issue associated with the risk that the MACE occurs based on the respective difference meeting the respective difference threshold, wherein the indication of the risk that the MACE occurs comprises an indication of the type of issue.

Example 18. The method of example 17, wherein the type of issue includes at least one of a coronary issue, a structural issue, or a conductive issue.

Example 19. The method of any of examples 12-18, wherein the indication comprises a timeframe of the risk of the MACE.

Example 20. The method of any of examples 12-19, wherein the respective SMA comprises a mean, median, or mode of the respective physiological parameter over an SMA period, the SMA period being a range measured in seconds, hours, days, months, or years.

Example 21. The method of any of examples 12-20, wherein the respective FMA comprises a mean, median, or mode of the respective physiological parameter over an FMA period, the FMA period being a range measured in seconds, hours, days, months, or years.

Example 22. The method of any of claims 12-21, wherein the SMA is determined over an SMA period and the FMA is determined over an FMA period, and wherein the FMA period is shorter than the SMA period.

Example 23. A non-transitory computer-readable storage medium storing instructions, which when executed, cause processing circuitry to: obtain one or more signals indicative of one or more respective physiological parameters; determine respective values of the one or more respective physiological parameters based on the one or more signals; determine a respective slow-moving average (SMA) of a first subset of the respective values of the one or more respective physiological parameters; determine a respective fast-moving average (FMA) of a second subset of the respective values of the one or more respective physiological parameters; determine that a respective difference between the respective FMA and the respective SMA meets a respective difference threshold; and based on the respective difference meeting the respective difference threshold, at least one of: a) set a flag indicative a risk that a major adverse cardiac event (MACE) occurs; or b) generate for output an indication of a risk that a MACE occurs.

Various examples have been described. These and other examples are within the scope of the following claims.

1. A system comprising:
memory configured to store physiological parameters of a patient; and
processing circuitry communicatively coupled to the memory, the processing circuitry being configured to:
obtain one or more signals indicative of one or more respective physiological parameters;
determine respective values of the one or more respective physiological parameters based on the one or more signals;
determine a respective slow-moving average (SMA) of a first subset of the respective values of the one or more respective physiological parameters;
determine a respective fast-moving average (FMA) of a second subset of the respective values of the one or more respective physiological parameters;
determine that a respective difference between the respective FMA and the respective SMA meets a respective difference threshold; and
based on the respective difference meeting the respective difference threshold, at least one of: a) set a flag indicative a risk that a major adverse cardiac event (MACE) occurs; or b) generate for output an indication of a risk that a MACE occurs.

2. The system of claim 1, wherein the processing circuitry is further configured to:
determine that a plurality of respective differences between respective FMAs and respective SMAs meet respective difference thresholds;
set respective flags indicative of each of the plurality of respective differences meeting the respective difference thresholds; and
generate for output an indication of a risk that a MACE occurs based on at least two of the respective flags.

3. The system of claim 1, wherein the one or more respective physiological parameters comprise at least one of a glucose level, a glucose time in range, a respiration rate, SpO2 blood oxygen saturation, blood oxygen perfusion index, heart rate, heart rate variability, night heart rate, blood pressure, pulse pressure, mean arterial pressure, radial arterial pressure, central venous pressure, pulse wave velocity or activity level.

4. The system of claim 1, further comprising one or more respective sensors, the one or more respective sensors being configured to sense the one or more respective physiological parameters.

5. The system of claim 4, wherein the one or more respective sensors comprise at least one of an implantable sensor or a wearable sensor.

6. The system of claim 1, wherein the processing circuitry is further configured to determine a type of issue associated with the risk that the MACE occurs based on the respective difference meeting the respective difference threshold, wherein the indication of the risk that the MACE occurs comprises an indication of the type of issue.

7. The system of claim 6, wherein the type of issue includes at least one of a coronary issue, a structural issue, or a conductive issue.

8. The system of claim 1, wherein the indication comprises a timeframe of the risk of the MACE.

9. The system of claim 1, wherein the respective SMA comprises a mean, median, or mode of the respective physiological parameter over an SMA period, the SMA period being a range measured in seconds, hours, days, months, or years.

10. The system of claim 1, wherein the respective FMA comprises a mean, median, or mode of the respective physiological parameter over an FMA period, the FMA period being a range measured in seconds, hours, days, months, or years.

11. The system of claim 1, wherein the SMA is determined over an SMA period and the FMA is determined over an FMA period, and wherein the FMA period is shorter than the SMA period.

12. A method comprising:
obtaining, by processing circuitry, one or more signals indicative of one or more respective physiological parameters;
determining, by the processing circuitry, respective values of the one or more respective physiological parameters based on the one or more signals;
determining, by the processing circuitry, a respective slow-moving average (SMA) of a first subset of the respective values of the one or more respective physiological parameters;
determining, by the processing circuitry, a respective fast-moving average (FMA) of a second subset of the respective values of the one or more respective physiological parameters;
determining, by the processing circuitry, that a respective difference between the respective FMA and the respective SMA meets a respective difference threshold; and
based on the respective difference meeting the respective difference threshold, at least one of: a) setting by the processing circuitry, a flag indicative a risk that a major adverse cardiac event (MACE) occurs; or b) generating by the processing circuitry, for output an indication of a risk that a MACE occurs.

13. The method of claim 12, further comprising:
determining, by the processing circuitry, that a plurality of respective differences between respective FMAs and respective SMAs meet respective difference thresholds;
setting, by the processing circuitry, respective flags indicative of each of the plurality of respective differences meeting the respective difference thresholds; and
generating, by the processing circuitry, for output an indication of a risk that a MACE occurs based on at least two of the respective flags.

14. The method of claim 12, wherein the one or more respective physiological parameters comprise at least one of a glucose level, a glucose time in range, a respiration rate, SpO2 blood oxygen saturation, blood oxygen perfusion index, heart rate, heart rate variability, night heart rate, blood pressure, pulse pressure, mean arterial pressure, radial arterial pressure, central venous pressure, pulse wave velocity, or activity level.

15. A non-transitory computer-readable storage medium storing instructions, which when executed, cause processing circuitry to:
obtain one or more signals indicative of one or more respective physiological parameters;
determine respective values of the one or more respective physiological parameters based on the one or more signals;
determine a respective slow-moving average (SMA) of a first subset of the respective values of the one or more respective physiological parameters;
determine a respective fast-moving average (FMA) of a second subset of the respective values of the one or more respective physiological parameters;
determine that a respective difference between the respective FMA and the respective SMA meets a respective difference threshold; and
based on the respective difference meeting the respective difference threshold, at least one of: a) set a flag indicative a risk that a major adverse cardiac event (MACE) occurs; or b) generate for output an indication of a risk that a MACE occurs.

16. The method of claim 12, further comprising sensing, by one or more respective sensors, the one or more respective physiological parameters.

17. The method of claim 16, wherein the one or more respective sensors comprise at least one of an implantable sensor or a wearable sensor.

18. The method of claim 12, further comprising determining, by the processing circuitry, a type of issue associated with the risk that the MACE occurs based on the respective difference meeting the respective difference threshold, wherein the indication of the risk that the MACE occurs comprises an indication of the type of issue.

19. The method of claim 18, wherein the type of issue includes at least one of a coronary issue, a structural issue, or a conductive issue.

20. The method of claim 12, wherein the indication comprises a timeframe of the risk of the MACE.
